(19) United States
(12) Patent Application Publication
(10) Pub. No.: US 2009/0104650 A1
(43) Pub. Date: Apr. 23, 2009

(54) Infectious diseases testing of menstrual fluid, endometrial/menstrual cells, amniotic fluid, umbilical cord blood or other samples

(75) Inventors: Mercedes A. Walton, Mendham, NJ (US); Julie G. Allickson, Odessa, FL (US)

Correspondence Address: DILWORTH PAXSON LLP, 1500 Market Street, Suite 3500 E, PHILADELPHIA, PA, 19102, US

(73) Assignee: Cryo-Cell International, Inc., Oldsmar, FL (US)

(21) Appl. No.: 12/283,844

(22) Filed: Sep. 15, 2008

Related U.S. Application Data
(60) Provisional application No. 60/993,748, filed on Sep. 14, 2007.

(51) Int. Cl. C12Q 1/02; G01N 33/68

(52) U.S. Cl. 435/29; 436/86

(57) ABSTRACT

Methods are provided for obtaining and testing or analyzing a non-venous and non-arterial puncture human fluid or cell sample or human body fluid or cell sample to detect the presence of at least one infectious disease. The sample may be menstrual fluid, endometrial menstrual cells, umbilical cord blood, or amniotic fluid. Confirmatory testing of a corresponding arterial or venous blood sample for comparison to the test results for the non-venous and non-arterial human fluid or cell sample may be performed. The testing may comprise a screening test or a confirmatory test.

### CROSS-REFERENCE TO RELATED APPLICATIONS

This application claims the priorities of U.S. Provisional Patent Application Ser. No. 60/993,748, filed Sep. 14, 2007, entitled “Infectious Disease Testing of Menstrual Fluid, Endometrial/Menstrual Cells, Amniotic Fluid, Umbilical Cord Blood or Other Samples,” and U.S. Provisional Patent Application Ser. No. ______, filed Sep. 9, 2008, entitled “Infectious Disease Testing of Menstrual Fluid, Endometrial/Menstrual Cells, Amniotic Fluid, Umbilical Cord Blood or Other Samples,” the entireties of which are incorporated herein by reference.

### FIELD OF THE INVENTION

The invention relates generally to infectious disease testing and specifically to infectious disease testing of samples of bodily fluid, tissues and/or cells or cellular components obtained or procured from, for example, any one of a specimen of menstrual fluid, endometrial menstrual cells, umbilical cord blood, or amniotic fluid.

### BACKGROUND OF THE INVENTION

There are many different types of infectious diseases in humans caused by viruses, bacteria and other agents. By way of non-limiting example, infectious diseases include, but are not limited to, Hepatitis A, Hepatitis B, Hepatitis C, Cytomegalovirus, Human T-cell Lymphotropic Virus Type 1, Human T-cell Lymphotropic Virus Type II, Human Immunodeficiency Virus Type 1, Human Immunodeficiency Virus Type II, West Nile Virus, Trypansoma cruzi, Syphilis, and Treponema pallidum. Several tests have been developed to determine whether a human has an infectious disease.

Infectious disease testing involves a wide array of test methodologies used to evaluate the presence and absence of infectious agents in a specimen collected from a human. Forms of enzyme immunoassays are relied upon for methods for infectious disease testing of venous and/or arterial blood samples. For example, testing methods may incorporate the enzyme immunoassay (EIA) and enzyme-linked immunosorbent assay (ELISA). The EIA and ELISA tests are used to detect and to quantify antigens and antibodies present in a venous and/or arterial blood sample. Because sensitivities of most enzyme immunoassays are high, they are typically used for screening samples for the presence of infectious diseases.

Many infectious disease tests employing enzyme immunoassay methods use a solid phase of an inactivated infectious agent coated onto micro wells and a detection platform. The solid phase test systems employ detection methods based on the adherence of red cell antibodies on the inactivated agent's antigen coated onto the surface of microtitration wells on a microtiter plate. The test procedure may be either a two or three step solid phase for red cell adherence tests carried out in the microtitration wells coated with the inactivated agent's antigen. Serum or plasma samples obtained from a venous or arterial blood specimen are added to the antigen coated wells. The serum or plasma samples are incubated with antibodies specific for the antigen that bind to the immobilized antigen. Unbound antibodies are washed from the wells and replaced with a suspension of coated indicator cells. A test result is considered positive when the usual migration of the indicator red cells to the bottom of the well is impeded by bridges formed between the red cells and the antigen-bound antibodies. The result causes the indicator red cells to adhere over the surface of the microtitration well. A test result is considered negative when there is an absence of antigen-antibody bridges allowing the indicator red cells to pellet to the bottom of the well as a packed, well-defined cell button. For the tests with multiple phases, the phases represent stepwise sample incubations, washings, and resulting assessments. In addition, many tests, whether for a virus, bacteria or other infectious agents, may use a calorimetric indicator to denote the test results.

Regardless of the test used for infectious disease testing, the specimen or sample type for testing is prescribed. Infectious disease screening tests licensed by the Food and Drug Administration (FDA) prescribe specific types of specimens to be used with licensed test kits. These specimens are serum, plasma, or cadaveric serum specimens which are obtained from venous or arterial puncture. Certain kit inserts expressly preclude use of a kit for testing saliva/oral fluid or urine samples.

Developments in the stem cell industry have identified new sources of stem cells from body fluids and tissue sources. For example, stem cells may be harvested from menstrual fluid, amniotic fluid, and umbilical cord blood. Infectious disease tests and assays are not available for use in infectious disease testing of menstrual fluid and endometrial/menstrual cells obtained from menstrual fluid, amniotic fluid samples, and/or umbilical cord blood.

Menstrual fluid, endometrial/menstrual cells and umbilical cord blood is a readily available specimen source that may be used for infectious disease testing. An amniotic fluid specimen would be a by-product of a previously-collected amniotic fluid sample used for another primary test assessing another parameter. Umbilical cord blood is another specimen that has been shown to be easily procured and provides a suitable amount of sample for testing. Indeed, the process of procuring endometrial/menstrual fluid and/or cells during the menstrual cycle, amniotic fluid from a previously-collected sample, and umbilical cord blood generally imparts little to no risk to the donor associated with collection techniques.

Menstrual fluid, endometrial/menstrual cells, amniotic fluid samples, and/or umbilical cord blood specimens are beneficial because each type of sample may serve as a single source for infectious disease testing. A single source sample does not require analysis of any other comparative sample, and its does not require analysis of an indirect sample. As an example of such indirect testing, and under current protocols in the cord blood industry, the presence or absence of infectious diseases in umbilical cord blood is determined indirectly from the results of testing of the mother's venous blood sample. A correlation of the infectious disease test results is made from the mother's blood relating to the umbilical cord blood. The cord blood is never directly tested.

The present invention provides methods, processes, and systems for direct testing of umbilical cord blood, menstrual fluid, endometrial/menstrual cellular suspensions, amniotic fluid, and/or other bodily fluids, tissues or cells for the present of infectious diseases or markers associated with infectious diseases such an infectious disease antigens and human antibodies created in an immune response to an infectious disease. Use of a single source specimen of umbilical cord blood, menstrual fluid, endometrial/menstrual cells, amniotic fluid, and/or other bodily fluids, tissues or cells samples provides for such direct infectious disease testing.

Accordingly, there is a present need for methods and processes for testing specimens of menstrual fluid, endometrial/menstrual cells suspensions, amniotic fluid, and/or umbilical cord blood for infectious disease. Thus, there is a need for methods and processes for infectious disease testing of these types of biological samples.

### SUMMARY OF THE INVENTION

The present provides methods and processes for infectious disease testing of menstrual fluid, endometrial/menstrual cells, amniotic fluid, and/or umbilical cord blood.

The invention includes methods and processes for procuring and processing blood, fluids, cells, and tissues obtained during menstruation, and includes the testing for the presence of infectious diseases and agents in the menstrual fluid and/or endometrial/menstrual cells in suspension. For example, the invention comprises methods and processes for procuring and processing blood, fluids, and tissues obtained during menstruation, and testing or analyzing the blood, fluid, and/or tissues to determine the presence of any infectious disease and infectious agents in the menstrual fluid and/or endometrial/menstrual cells.

The invention includes methods and processes for processing amniotic fluid obtained as a by-product of amniotic fluid sampling collected for the primary purpose of other clinical or research assessments, and includes the testing for the presence of infectious diseases and agents in the sample of amniotic fluid. For example, the invention comprises methods and processes for processing amniotic fluid samples obtained as a by-product of amniotic fluid samples collected for the primary purpose of other clinical or research assessments, and testing or analyzing the blood, fluid, and/or tissues to determine the presence of any infectious disease and infectious agents in the amniotic fluid.

The invention includes methods and processes for processing umbilical cord blood obtained at childbirth, and includes the testing for the presence of infectious agents/diseases in the umbilical cord blood. For example, the invention comprises methods and processes for procuring and processing umbilical cord blood obtained at childbirth, and testing or analyzing the blood, fluid, and/or tissues to determine the presence of any infectious disease and infectious agents in the umbilical cord blood.

In an embodiment, the present invention provides a method for analyzing a non-venous and non-arterial puncture human fluid or cell sample to detect the presence of at least one infectious disease. The method comprises the step of first obtaining a sufficient volume of the non-venous and non-arterial puncture human fluid or cell sample. The non-venous and non-arterial puncture human fluid or cell sample may be procured from any one of a specimen of menstrual fluid, endometrial menstrual cells, umbilical cord blood, or amniotic fluid. The method comprises the following step of testing the sufficient volume of the non-venous and non-arterial human fluid or cell sample for at least one infectious disease.

The present invention provides additional steps of performing confirmatory testing of an arterial or venous blood sample obtained from a human and comparing confirmatory testing results to test results for the non-venous and non-arterial puncture human fluid or cell sample.

The present invention provides that the infectious disease tested for in the non-venous and non-arterial human fluid or cell sample comprises any one of Hepatitis A, Hepatitis B, Hepatitis C, Cytomegalovirus, Human T-cell Lymphotropic Virus Type 1, Human T-cell Lymphotropic Virus Type II, Human Immunodeficiency Virus Type 1, Human Immunodeficiency Virus Type II, West Nile Virus, Trypansoma cruzi, Syphilis, and Treponema pallidum. Biological samples may be tested for other infectious diseases.

The present invention provides for testing that may comprise analyzing the biological sample for at least one form of antigen or antibody associated with the presence of at least one infectious disease in a human.

In another embodiment, the present invention provides a method for direct testing of a menstrual fluid sample from a human for the presence of at least one infectious disease. The method comprises isolating a sufficient volume of the menstrual fluid sample from a pre-collected specimen. The method comprises analyzing the menstrual fluid sample for the presence of at least one or more infectious diseases.

The present invention provides that the infectious diseases that may be directly tested for in the menstrual fluid sample comprises any one of Hepatitis A, Hepatitis B, Hepatitis C, Cytomegalovirus, Human T-cell Lymphotropic Virus Type 1, Human T-cell Lymphotropic Virus Type II, Human Immunodeficiency Virus Type 1, Human Immunodeficiency Virus Type II, West Nile Virus, Trypansoma cruzi, Syphilis, and Treponema pallidum.

The present invention provides that the step of analyzing the menstrual fluid sample comprises determining the presence of at least one form of antigen associated with an infectious disease or an antibody created by a human's immune response to the presence of an infectious disease in the human's body.

In a further embodiment, the present invention provides a method for direct testing of a human body fluid or cell sample for the presence of at least one infectious disease. The method provides preparing a sufficient volume of human body fluid or cell sample for analysis. The method also provides analyzing human body fluid or cell sample for antigens or antibodies associated with an infectious disease.

The present invention provides that the human body fluid or cell sample may be procured from any one of a specimen of menstrual fluid, endometrial menstrual cells, umbilical cord blood, or amniotic fluid.

The present invention provides that the infectious disease tested for in the human body fluid or cell sample comprises any one of Hepatitis A, Hepatitis B, Hepatitis C, Cytomegalovirus, Human T-cell Lymphotropic Virus Type 1, Human T-cell Lymphotropic Virus Type II, Human Immunodeficiency Virus Type 1, Human Immunodeficiency Virus Type II, West Nile Virus, Trypansoma cruzi, Syphilis, and Treponema pallidum. The human body fluid or cell sample may be tested for other infectious diseases.

The present invention provides that the testing may comprise a screening test or a confirmatory test.

The present invention provides that the testing may comprise analyzing the human body fluid or cell sample for the presence and/or quantity of an antigen produced by an infectious disease or the presence and/or quantity of a human antibody associated with the presence of an infectious disease in a human.

The present invention provides an additional step of correlating the screening test results with the confirmatory test results. The screening test results may comprise test results obtained by the embodiments of the present invention for non-venous and non-arterial puncture human fluid or cell samples, menstrual fluid samples, and human body fluid or cell samples. The confirmatory test results may be obtained from additional testing of non-venous and non-arterial human fluid or cell samples, menstrual fluid samples, and human body fluid or cell samples or alternatively corresponding venous or arterial blood samples.

The present invention provides for determining the presence or absence of infectious disease antigens or human antibodies created by an immune response to infectious disease antigens.

### BRIEF DESCRIPTION OF THE DRAWINGS

FIG. 1 shows a flow chart depicting an overview of an embodiment of a method to procure and to process menstrual blood, fluids, tissue and cells;

FIG. 2 shows a flow chart depicting an overview of an embodiment of a method to procure and process menstrual blood, fluids, tissue and cells;

FIG. 3 shows a flow chart depicting another embodiment for procuring and processing menstrual blood, fluids, tissue and cells and testing for infectious diseases;

FIG. 4 shows a flow chart depicting an overview of a method of the present invention for procuring and processing umbilical cord blood and testing for infectious diseases; and

FIG. 5 shows a flow chart depicting an overview of a method of the present invention for procuring and processing amniotic fluid and testing for infectious diseases.

### DESCRIPTION OF THE EMBODIMENTS OF THE INVENTION

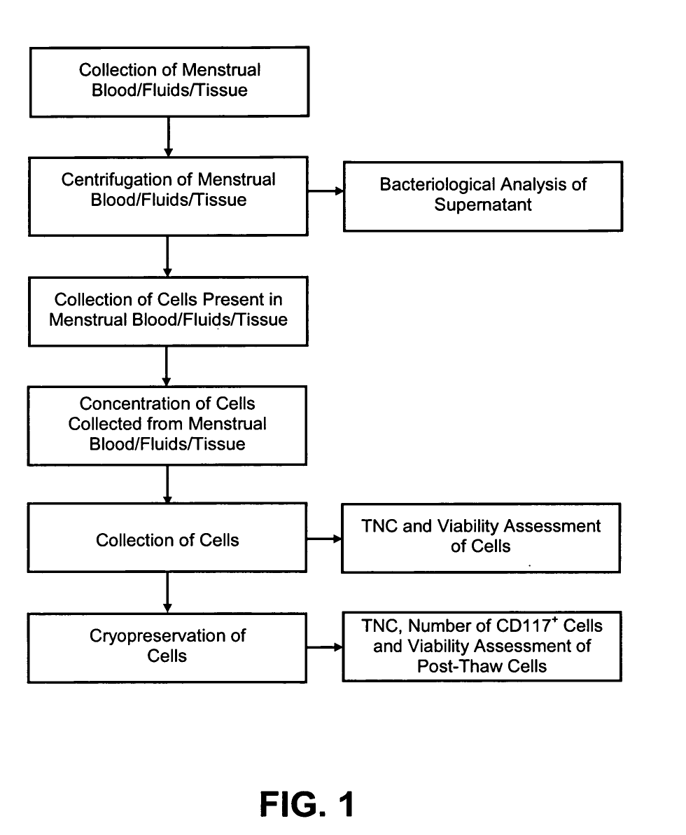

In reference to FIGS. 1 through 5, infectious disease testing methods and processes for menstrual fluid, endometrial/menstrual cells, amniotic fluid, and/or umbilical cord blood are provided by the present invention.

Methods are provided for obtaining a sample of menstrual fluid, endometrial/menstrual cells, amniotic fluid samples, umbilical cord blood or other bodily fluid or tissue. The methods comprise the further step of testing a suitable volume of sample with infectious disease testing methods. The infectious disease testing methods may be selected for testing for infectious diseases of interest. The infectious disease testing methods may be commercial tests.

A method is provided for analyzing a non-venous and non-arterial puncture human fluid or cell sample to detect the presence of at least one infectious disease. The method comprises first obtaining a sufficient volume of the non-venous and non-arterial puncture human fluid or cell sample. The non-venous and non-arterial human fluid or cell sample may be procured from any one of a specimen of menstrual fluid, endometrial menstrual cells, umbilical cord blood, or amniotic fluid.

The method comprises the additional step of testing the sufficient volume of the non-venous and non-arterial human fluid or cell sample for an infectious disease. The testing may be focused on analyzing the biological sample for an antigen or antibody associated with the presence of an infectious disease in a human. The infectious disease testing may be in the nature of a screening test or a confirmatory test.

The method may comprise an additional step of performing confirmatory testing of an arterial or venous blood sample obtained from a human and comparing confirmatory testing results to test results for the non-venous and non-arterial human fluid or cell sample. The test results are reported to the patient, sample donor, health care provider, and/or stem cell banking provider.

Methods are provided for direct testing of a menstrual fluid sample from a human for the presence of at least one infectious disease. The method comprises isolating a sufficient volume of the menstrual fluid sample from a pre-collected specimen. The method comprises analyzing the menstrual fluid sample for the presence of an infectious disease. The analysis of the menstrual fluid sample may comprise a screening test or a confirmatory test. The menstrual fluid sample is analyzed to determine the presence or absence of an antigen associated with an infectious disease or an antibody created by a human's immune response to the presence of an infectious disease.

Methods are provided for direct testing of a human body fluid or cell sample for the presence of at least one infectious disease. The method provides preparing a sufficient volume of human body fluid or cell sample for analysis. The human body fluid or cell sample may be procured from any one of a specimen of menstrual fluid, endometrial menstrual cells, umbilical cord blood, or amniotic fluid. The method also provides analyzing human body fluid or cell sample for antigens or antibodies associated with an infectious disease. The analysis may comprise a screening test or a confirmatory test. The testing is focused on analyzing the human body fluid or cell sample for the presence and/or quantity of an antigen produced by an infectious disease or the presence and/or quantity of a human antibody associated with the presence of an infectious disease in a human.

The methods of the present invention provide for infectious disease testing. Infectious diseases that are tested for by way of the present invention include, but are not limited to, Hepatitis A, Hepatitis B, Hepatitis C, Cytomegalovirus, Human T-cell Lymphotropic Virus Type 1, Human T-cell Lymphotropic Virus Type II, Human Immunodeficiency Virus Type 1, Human Immunodeficiency Virus Type II, West Nile Virus, Trypansoma cruzi, Syphilis, and Treponema pallidum. The present invention may test for other infectious diseases.

Sources of Fluid or Cell Sample for Infectious Disease Testing

The present invention provides for several sources of human fluid or cell samples for infectious disease testing. The sample source may be characterized as a non-venous and non-arterial puncture human fluid or cell specimen. The sample source may also be characterized as a human body fluid or cell sample. In particular, the sample source comprises menstrual fluid that may be menstrual flow collected during menstruation, amniotic fluid collected during amniocentesis, or umbilical cord blood collected during birth. Specimens of each of these sample sources that are initially collected may be tested for infectious diseases. Alternatively, the sample sources may be processed to obtain concentrated volumes of plasma, serum, and/or cellular suspensions that may be tested. If needed for infectious disease testing, the cells of the cellular suspension may be lysed to obtain cellular components for infectious disease testing.

Menstrual Fluid

Referring now to FIGS. 1 through 3, the invention comprises methods for procuring menstrual fluid specimens. Menstrual fluid specimens comprise menstrual blood, fluids, cells, and tissues obtained during menstruation. A menstrual fluid specimen may be collected from the menstrual fluid and then analyzing or tested to determine the presence of at least one infectious disease antigen associated with an infectious disease or the presence of human antibodies produced by an immune response to an infectious disease that are present in the menstrual fluid.

Menstrual fluid samples may be collected in a variety of different ways for infectious disease testing. In an embodiment, the menstrual flow may be collected according to the processes and methods described in U.S. patent application Ser. No. 12/074,423, entitled “Procurement, Isolation, and Cryopreservation of Endometrial/Menstrual Cells” filed Mar. 2, 2008 which is incorporated herein in its entirety by reference. Other processes and methods for collecting a menstrual fluid sample may be used so long as a sufficient volume of menstrual flow sample is collected to perform the step of testing the menstrual fluid sample for the presence of an infectious disease.

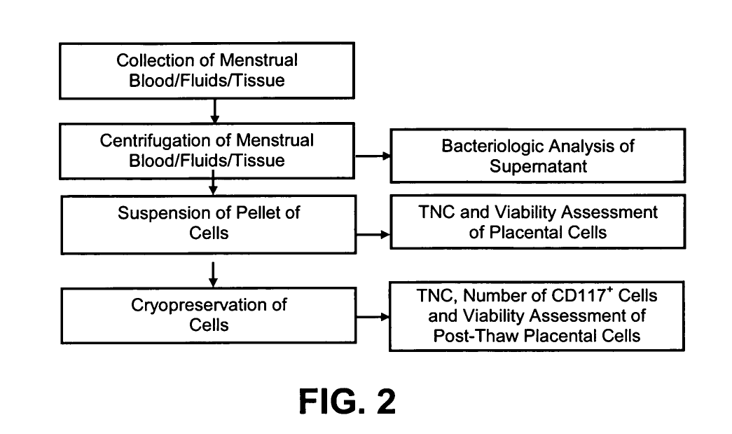

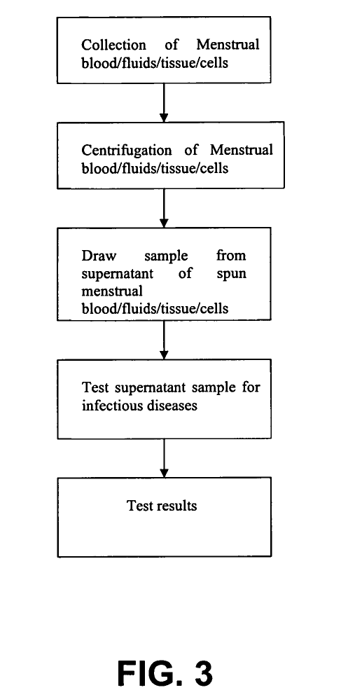

In an embodiment, a donor may use a procurement kit to collect a may comprise media tubes that contain collection media in a rack covered in Parafilm, at least one collection device and instructions (i,e., INSTEAD as described in FIG. 2 or the DIVA cup as described in FIG. 3), antiseptic cleaning pads, small plastic bags, small biohazard bag (to place specimens in for return to laboratory), Parafilm, nanocooler, and a collection form. The collection kit may remain at room temperature prior to use. Alternatively, the collection kit may be maintained at a refrigerated temperature of about 1° C. to about 10° C. prior to use.

The menstrual flow sample may be collected in a collection cup designed for menstrual flow collection. The collection of menstrual flow may take place on one of the heaviest days of the donor's menstrual period which may be the first or second day. The collection cup may be sterilized prior to use and the donor should follow the manufacturer's instructions for insertion of the collection cup into the vagina.

The collection cup may be inserted into the vagina. The general area around the vagina may be cleansed with an aseptic cleaning pad prior to insertion of the collection cup. The collection cup may remain inside the vagina for about up to 3 hours or less or any other suitable amount of time necessary to collect a menstrual flow sample in a sufficient volume to allocate some of the menstrual flow sample for infectious disease testing. After a suitable amount of time has passed with the collection cup positioned inside the vagina, a donor may then remove the cup and pour the contents comprising menstrual flow contents into a 50 milliliter collection tube holding collection media. The collection media may comprise about 10 milliliters of an isotonic solution with an anticoagulant. For example, but not as a limitation, Hank's Balanced Salts Solution may be used as an isotonic solution, and Heparin (10 units per milliliter) may be used as an anticoagulant. Other collection media may be used including, but not limited to, the collection media described in U.S. patent application Ser. No. 12/074,423. The collection tube should be closed and sealed.

Multiple menstrual flow samples may be collected using a new collection cup each time. If multiple menstrual flow samples are collected, the entire sample of menstrual flow in a single collection cup should be placed in one 50 milliliter collection tube containing collection media. One collection tube should be used for each sample of menstrual flow. Whether a single sample or multiple samples are collected, the sample or samples may be maintained at room temperature (about 15° C. to about 25° C.) prior to shipment to the laboratory so long as the sample or samples arrive at the laboratory within about 24 hours to about 72 hours of collection. Alternatively, and in cases where multiple samples are collected, collection tubes containing samples may be refrigerated at about 1° C. to about 10° C. until all samples have been collected prior to shipment. Alternative shipping and handling methods and processes for menstrual fluid are provided in U.S. patent application Ser. No. 12/074,423 and may be used in the present invention. The package containing the menstrual flow sample or samples may be maintained at room or ambient air temperature (about 15° C. to about 25° C.) throughout shipment and should arrive at the laboratory within about 24 hours of collection and may be refrigerated upon arrival at the laboratory. Alternatively, the package containing the menstrual flow sample or samples may be maintained at a temperature of about 1° C. to about 10° C. throughout shipment.

A peripheral blood sample may also be collected from the donor for screening or comparative infectious disease testing purposes. The peripheral blood sample may be collected according to standard phlebotomy techniques in an evacuated blood collection system holder and the evacuated tube with EDTA (dipottasium salt of ethylenediaminetetraacetic acid). The peripheral blood sample may also be shipped along with the menstrual flow sample to a laboratory as previously described.

The present invention comprises the step of analyzing and determining the presence of any infectious disease and infectious agents in the menstrual flow sample. Upon arrival of the menstrual flow sample or samples at a laboratory, the collection tube, or tubes if multiple menstrual flow samples are collected, are disinfected with IPA or Cavicide, for example, and transferring each collection tube to a biologically safety cabinet under sterile conditions. Each collection tube may be placed on ice or maintained at a temperature between about 1° C. to about 10° C. throughout processing in preparation for aliquoting a sample into a transfer tube for infectious disease testing.

The menstrual flow sample may be processed by removing an aliquot of about 5 milliliters from the sample and placing the sample in a transfer tube, such as a 6 milliliter transfer tube or other tube suitably size to hold a sufficient volume of sample for testing. As an alternative and as described in FIG. 3, the menstrual flow sample may be centrifuged at about 2000 RPM for about 4° C. Centrifugation may also take place at room temperature. Once the centrifugation is completed, a 10 milliliter sample may be collected from the supernatant and placed in a transfer tube.

The transfer tube with either an uncentrifuged menstrual flow sample or supernatant from a centrifuged menstrual flow sample may be labeled and prepared for infectious disease testing. The transfer tube may be maintained at room temperature if infectious disease testing will occur at a time period less than about 7 days. Alternatively, the transfer tube may be refrigerated at about 1° C. to about 10° C. for up to about 7 days prior to infectious disease testing the transfer tube may be maintained at a temperature between about 1° C. to about 10° C. throughout shipment to an off site infectious disease testing service provider. Alternatively, the transfer tube may be shipped at room temperature.

In a further embodiment, a pre-processing sample of supernatant obtained from a menstrual flow sample specimen that was procured and processed in connection with methods and processes of menstrual stem cell collection, such as those generally outlined and described in FIGS. 1 and 2 or as described in U.S. patent application Ser. No. 12/074,423 may be obtained for infectious disease testing.

In another embodiment, a suitable volume of a post-processing menstrual flow sample obtained according to the methods and processes of U.S. patent application Ser. No. 12/074,423 may be obtained for infectious disease testing.

The transfer tube containing an aliquot of menstrual flow, centrifuged menstrual flow, supernatant from a pre-processing sample or from post-processing sample may be tested for infectious disease markers of interest. For example, an order form for an infectious disease testing may be completed to order infectious marker testing for infectious diseases of interest if testing is to occur off site by a laboratory or a service provider that performs infectious disease testing services. Alternatively, the transfer tube containing the menstrual flow sample may be tested for infectious diseases at the menstrual flow sample-receiving laboratory. A transfer tube may be refrigerated at about 1° C. to about 110° C. for up to about 7 days prior to infectious disease testing. A transfer tube may be maintained at a temperature between about 1° C. to about 10° C. throughout shipment to an off site infectious disease testing service provider. Alternatively, the transfer tube may be shipped at room temperature.

Alternative forms of collecting, transporting, preparing, and testing a menstrual flow sample are provided in U.S. patent application Ser. No. 12/074,423 and may be used for obtaining a menstrual flow sample for infectious disease testing according to the invention.

Umbilical Cord Blood Samples

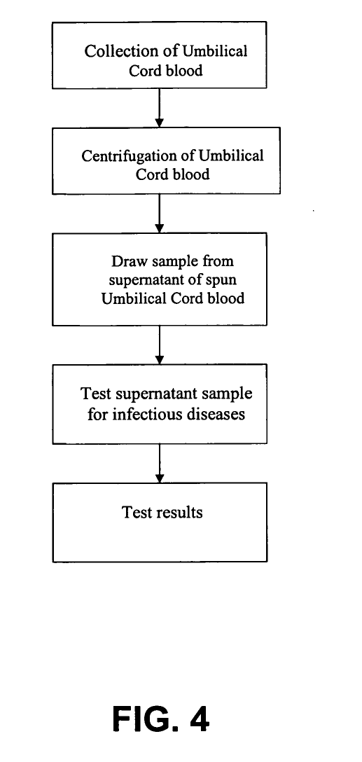

Referring to FIG. 4, the present invention provides methods and processes for obtaining an umbilical cord blood sample during at childbirth, and then analyzing or testing the umbilical cord blood sample to determine the presence of any infectious disease antigens or human antibodies resulting from an immune response to the infectious disease antigen in the umbilical cord blood sample.

Umbilical cord blood may be obtained at childbirth using any manner of umbilical cord blood collection methods. In an embodiment, a cord blood collection kit (U-Cord Collection Kit Contents: Blue Basin with Clear Top, Mother's Blood Draw Kit, DonorCare™ Needle Guard, Alcohol Wipes, Tincture of Iodine Swabstick, Baxter Blood Collection Bag (250 mL) with CPD anticoagulant and 16 gauge needle, C-Section Adapter Kit (Sterile), Plastic zip bag with adhesive backing and absorbent towels) may be used in accordance with manufacturer's instructions. For example, cord blood collection for a vaginal delivery may occur by double clamping the cord and cutting the umbilical cord. The blue plastic DonorCare needle guard may be clamped around the collection tubing. After delivery of a baby(s) and prior to expulsion of a placenta, about 4-6″ of umbilical cord may be cleansed with an alcohol wipe followed by a Tincture of Iodine swab. For maximum volume, a needle may be inserted at an insertion site just above the clamp that remains on the umbilical cord. Using gravity, collect as much blood as possible into the bag. At least about 80 mL (including 35 mL of anticoagulant) of collection may be needed for processing. When collection is complete, the bag may be at least ⅓ full. When collection is completed, the blood may be milked in the tubing down into the bag. The bag may be gently inverted several times to mix the umbilical cord blood and CPD anticoagulant. For multiple births, individual cord blood kits should be used for each baby.

If collection of an umbilical cord blood sample occurs after cesarean delivery, a C-Section Adapter Kit may be used for double clamping the cord after birth and inserting a needle into the umbilical vein to collect umbilical cord blood. As much blood as is possible may be collected in the bag. At least about 80 mL (including 35 mL of anticoagulant) of collection may be needed for processing. Gently invert bag several times to mix the cord blood and CPD anticoagulant. For multiple births, individual cord blood kits should be used for each baby.

If placenta is detached, the umbilical cord may be cleansed. A needle may be inserted into the umbilical vein attached to the placenta. The detached placenta may be elevated to facilitate collection of cord blood. At least about 80 mL (including 35 mL of anticoagulant) of collection may be needed for processing. For multiple births, individual cord blood kits should be used for each baby.

A collected umbilical cord blood sample may be prepared for shipment by wrapping a labeled cord blood bag with the absorbent towels. The cord blood bag may be placed in a large plastic zip bag that is closed. An adhesive backing may be removed from the large plastic zip bag then the bag may be pressed to bottom of a container to secure the collected blood sample for shipment. A lid may be placed on the container and secured with tape. AirNet or another suitable courier may be contacted within about 2 hours of birth(s) to arrange shipment. The cord blood shipment should arrive in a laboratory within at least about 48 hours of collection.

The cord blood bag should be transferred into a laboratory, sterilized, and prepared for processing in a biological safety cabinet. A sample of the cord blood may be drawn for infectious disease testing. Alternatively, a sample of cord blood may be obtained after removal of the red cells from the umbilical cord blood sample. The cord blood sample used for infectious disease testing may be about one milliliter of cord blood for a red cell sample and up to about three milliliters for a plasma sample.

The sample of cord blood will be transferred to a sterile collection tube coated with EDTA. The sample of cord blood or cord blood plasma may be transferred to two or more 6 milliliter tubes for testing. The tubes may or may not contain an anticoagulant, such a Heparin.

Amniotic Fluid

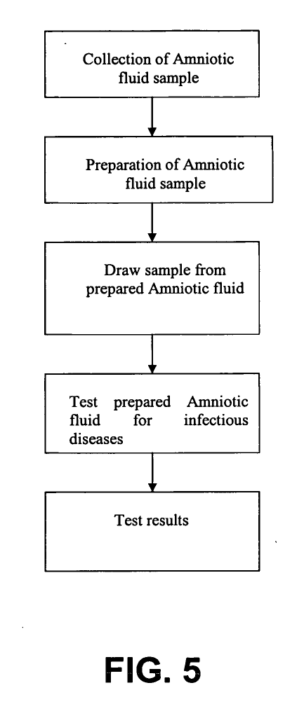

Referring to FIG. 5, the present invention comprises methods and processes for obtaining amniotic fluid samples as a by-product of amniotic fluid samples collected for the primary purpose of other clinical or research assessments, and analyzing or testing the amniotic fluid sample to determine the presence of any infectious disease antigens or human antibodies created by an immune response to the infectious disease antigens in the sample of amniotic fluid.

The invention provides processing amniotic fluid samples obtained as a by-product of amniotic fluid samples collected for the primary purpose of other clinical or research assessments. Amniotic fluid is collected as a medical procedure used for prenatal diagnosis, in which a small amount is extracted from the amnion around the developing fetus. Amniocentesis may be performed when there is enough fluid surrounding the fetus. The sample may be removed via a syringe and guidance via ultrasound. Amniocentesis may be performed as early as about 13 weeks of gestation, but may also be collected between about 15 weeks to about 20 weeks of gestation. When the amniotic fluid sample is collected by amniocentesis for genetic testing, about 10 milliliters of the amniotic fluid may be aliquoted into 2-6 milliliter collection tubes coated with EDTA, or other suitable anticoagulant for infectious disease testing.

Other Fluids, Tissues and Cells

As yet a further example, the invention comprises methods and processes for procuring and processing any other human bodily fluid, cell or tissue and analyzing and determining the presence of any infectious disease and infectious agents in the human bodily fluid, cell or tissue. For example, cellular suspensions of fetal placental cells and maternal placental cells may be tested for infectious diseases. The placental samples may be obtained by methods disclosed in U.S. Patent Application Publication No. 20080064098, entitled “Procurement, Isolation and Cryopreservation of Maternal Placental Cells,” published on Mar. 13, 2008 and U.S. Patent Application Publication No. 20080050814, entitled “Procurement, Isolation and Cryopreservation of Fetal Placental Cells,” published on Feb. 28, 2008. A pre-processing or post-processing maternal placental stem cell sample or a fetal placental stem cell sample, as described in the preceding published U.S. patent applications may be tested for the presence of an infectious disease antigen or human antibody created by an immune response to the infectious disease antigen present in either type of placental stem cell sample.

Infectious Disease Testing Methods

The methods and processes of the present invention also provide steps for analyzing or testing non-venous and non-arterial puncture human fluid or cells samples and human body fluid or cell samples to determine the presence of any infectious disease antigens or human antibodies created by an immune response to the infectious disease antigens. The sources of the non-venous and non-arterial puncture human fluid or cells samples and human body fluid or cell samples may comprise menstrual fluid that may be collected during menstruation, amniotic fluid collected during amniocentesis, or umbilical cord blood collected during birth.

The present invention provides that the sample of the non-venous and non-arterial puncture human fluid or cells samples or human body fluid or cell samples in the transfer tube is tested or analyzed for infectious diseases. According to the present invention, the transfer tube containing any one of menstrual flow, centrifuged menstrual flow, supernatant from a pre-processing sample or from post-processing sample, cord blood, cord blood plasma, amniotic fluid sample, pre-processing or post-processing maternal placental stem cell sample, or a fetal placental stem cell sample is analyzed or tested for the infectious disease of interest.

The present invention provides that infectious disease testing of any one of menstrual flow, centrifuged menstrual flow, supernatant from a pre-processing sample or from post-processing sample, cord blood, cord blood plasma, amniotic fluid sample, pre-processing or post-processing maternal placental stem cell sample, or a fetal placental stem cell sample may use commercially-available testing methods, other FDA approved, licensed or commercially acceptable infectious disease testing methodologies, or other testing methods for infectious diseases. Specimen volumes for infectious testing would be based on required volumes for commercially-available tests or as otherwise required.

The testing may be performed by any infectious disease testing laboratory. For example, an order form may be completed to order infectious marker testing for infectious diseases of interest for testing to occur off site by a laboratory or a service provider. Alternatively, the transfer tube containing the menstrual flow sample may be tested for infectious diseases at the laboratory where the body fluid or cell sample was shipped for processing. A transfer tube with a specimen may be refrigerated at about 1° C. to about 110° C. for up to about 7 days prior to infectious disease testing. A transfer tube may be maintained at a temperature between about 1° C. to about 110° C. throughout shipment to an off site infectious disease testing service provider. Alternatively, the transfer tube may be shipped at room temperature.

By way of non-limiting example, the infectious diseases that may be tested for in the specimens include, but are not limited to, Hepatitis C Virus, Hepatitis B Virus Core Antigen, Hepatitis B Surface Antigen, HIV-I/II, Cytomegalovirus, HTLV I/II, West Nile Virus, Syphilis, and Trepanome pallidum, and any other infectious diseases.

The specimen of menstrual flow, centrifuged menstrual flow, supernatant from a pre-processing sample or a post-processing sample, cord blood, cord blood plasma, amniotic fluid sample, pre-processing or post-processing maternal placental stem cell sample, or a fetal placental stem cell sample is analyzed by infectious disease testing methodologies to detect the presence of at least one or more infectious disease antigen and/or complementary human antibodies.

Hepatitis C

The infectious disease test for the Hepatitis C virus may be used to test for the presence of Hepatitis C virus (HCV). A test method used to detect HCV may be an ELISA test for the detection of human antibodies to Hepatitis C Virus (Anti-HCV) in human serum or plasma. The test utilizes microwells coated with recombinant hepatitis C virus encoded antigens as the solid phase. When enzyme substrate is applied, the presence of antigen or antibody can be detected by development of a colormetric end-product. A commercially-available test is the Ortho ELISA Test System. This test may be used to test a sample for infectious disease. Other HCV diagnostic tests may be used to test for HCV in specimens.

The assay procedure is a three-stage test carried out in a microwell coated with a combination of recombinant hepatitis C virus (rHCV) antigen (antigens: C22-3, c200 and NS5). In the first stage, a diluted test specimen of about 10 microliters is incubated for about 60 minutes in the test well. If antibody reactive to any of the three antigens is present, complexes will not be formed. In the subsequent washing step, unbound serum or plasma proteins will be removed. In the second stage, about 200 microliters of murine monoclonal antibody conjugated to horseradish peroxidase is added to the microwell. The conjugate binds specifically to the human IgG portion of the antigen-antibody complexes. If antigen-antibody complexes are not present, the unbound conjugate will be removed by subsequent washing. In the third stage, about 200 microliters of an enzyme detection system composed of o-phenylenediamine (OPD) and hydrogen peroxide is added to the test well. If bound conjugate is present, the OPD will be oxidized, resulting in a colormetric end-product. In this reaction, peroxidase is divalently oxidized by hydrogen peroxide to form an intermediate compound, which is in turn, reduced to its initial state by subsequent interaction with hydrogen ion donating OPD. The resulting oxidized form of OPD has an orange color. About 50 microliters of 4N Sulfuric acid is then added to stop the reaction. The color intensity depends on the amount of bound conjugate in the well, and, therefore, color intensity is a function of the concentration of anti-HCV present in the specimen. The color intensity is measured with a microwell reader (photometer) designed to measure light absorbance in a microwell. Results of the test are interpreted by the absorbance value of the color change, based on a determined cutoff absorbance value. Specimens with absorbance values less than the cutoff are considered non-reactive or negative for the Hepatitis C Virus and those above the cutoff absorbance value are considered reactive or positive for Hepatitis C Virus.

Hepatitis B Virus Core Antigen

Hepatitis B Virus Core Antigen (recombinant) is an enzyme-linked immunosorbent assay for the detection of antibody to Hepatitis B Virus Core Antigen (anti-HBc) in Human Serum or Plasma. A variety of serologic markers appear following infection with hepatitis B virus (HBV). The first marker to appear is usually hepatitis B surface antigen (HBsAg). Antibodies to hepatitis B core antigen (anti-HBc) appear next, and remain detectable following the clearance of HBsAg and into convalescence. The determination of anti-HBc in serum and plasma may be used as an aid to monitor the progress of HBV infection. Anti-HBc appears in virtually all individuals infected with HBV and is an accurate serological marker of recent and past infection. A commercially available test is the Ortho ELISA Test System. This test may be used to test a specimen for infectious disease. Other suitable tests may be used to test for Hepatitis B.

The enzyme-linked immunosorbent assay for detection of Hepatitis B Virus Core Antigen may be a three-stage test carried out in a microwell coated with recombinant-derived hepatitis B core antigen (HBc). In the first stage, a test specimen of about 10 microliters is placed directly in the test well containing specimen diluent and incubated for about 60 minutes. If anti-HBc is present in the specimen, antigen-antibody complexes will form on the microwell surface. If anti-HBc is not present, complexes will not form and the unbound serum or plasma proteins will be removed in the washing step. In the second stage, about 200 microliters of antibody conjugate is added to the test well and incubated for about 60 minutes. The antibody conjugate is a mixture of murine monoclonal antibodies specific for human IgG and IgM. The conjugate will bind specifically to the antibody portion of the antigen-antibody complexes. If antigen-antibody complexes are not present, the unbound conjugate will be removed by washing. In the third stage, an enzyme detection system composed of about 200 microliters of o-phenylenediamine (OPD) and hydrogen peroxide is added to the test well and incubated for about 60 minutes. If bound conjugate is present, the OPD will be oxidized, resulting in a colored end-product. 50 microliters of 4N Sulfuric acid is then added to stop the reaction. The color intensity depends on the amount of bound conjugate and therefore is a function of the concentration of anti-HBc present in the specimen. The color intensity is measured with a microwell reader. Results of the test are interpreted by the absorbance value of the color change, based on a determined cutoff absorbance value. Specimens with absorbance values less than the cutoff are considered non-reactive or negative for the Hepatitis B Virus Core Antigen. Those above the cutoff absorbance value are considered reactive or positive of Hepatitis B Virus Core Antigen.

Another possible test is a test for the antibody to Hepatitis B Surface Antigen (Murine Monoclonal) Peroxidase Conjugate antibody to HBsAg ELISA (Enzyme-Linked Immunosorbent Assay) is used for the detection of Hepatitis B Surface Antigen (HBsAg) in human serum or plasma. Such a test may be used as a screening test and an aid in diagnosis of potential hepatitis B infection. The test utilizes microwells coated with antibody to HBsAg as a solid phase. When enzyme substrate is applied, the presence of antigen or antibody can be detected by development of a colored end-product. A commercially available test is the Ortho Antibody to HBsAg ELISA. Other suitable tests may be used to test for Hepatitis B.

The test is a two-stage assay carried out in a microwell coated with antibody to HBsAg. In the first stage, working conjugate comprised of antibody to HBsAg, and diluted in conjugate diluent that is blue in color, is added to the test well. The test specimen is then added to the test well and a SOM (sample omission monitoring) read is performed. The plate is incubated for a specified length of time. If HBsAg is present in the specimen, it will bind to the antibody coated on the well and simultaneously bind to the conjugate to form immobilized antibody-HBsAg-conjugate complexes. If HBsAg is not present, these complexes will not be formed. Unbound serum or plasma proteins will be removed in the subsequent washing steps. In the second stage, an enzyme detection system composed of o-phenylenediamine (OPD) and hydrogen peroxide is added to the test well. If bound conjugate is present, the OPD will be oxidized, resulting in a colored end-product. In this reaction, peroxidase is divalently oxidized by hydrogen peroxide to form an intermediate compound, which is in turn, reduced to its initial state by subsequent interaction with hydrogen ion donating OPD. The resulting oxidized form of OPD has an orange color. Sulfuric acid is then added to stop the reaction. The color intensity depends on the amount of bound conjugate in the well. Therefore, color intensity is a function of the concentration of HBsAg present in the specimen. The color intensity may be measured with a microwell reader at 490 or 492 nm. Results of the test are interpreted by the absorbance value of the color change, based on a determined cutoff absorbance value. Specimens with absorbance values less than the cutoff are considered non-reactive or negative for the Hepatitis B Surface Antigen. Those above the cutoff absorbance value are considered reactive or positive of Hepatitis B Surface Antigen.

Human Immunodeficiency Virus-1/2

The test for the Human Immunodeficiency Virus (HIV) is a synthetic peptide enzyme immunoassay (EIA) for the detection of the antibody to Human Immunodeficiency Virus Types 1 and/or 2 (HIV-1 and HIV-2). The test uses microwells that are coated with a mixture of four peptides from the virus. A commercially-available test is the Bio-Rad HIV-1/HIV-2 EIA test kid. This test may be used to test a sample for infectious disease. Other suitable tests may be used to test for HIV-1/2.

Specimens are evaluated for the presence of HIV-1 and HIV-2 antibodies by interaction with the adsorbed peptides in the wells. Specimens to be tested are diluted in specimen diluent, added to each well, and the plates containing the wells are incubated and washed. If antibodies to either HIV-1 or HIV-2 are present, they bind to the adsorbed antigen and are not removed by washing. The working conjugate solution, peroxidase-labeled goat anti-human immunoglobulin, is then added to the wells and will bind to the antibody-antigen complex, if present. Unbound conjugate is removed by a wash step. Next, working chromogen solution is added to the plate and allowed to incubate. A blue or blue-green color develops in proportion to the amount of antibody that has been bound to the antigen-coated plate. The enzyme reaction is stopped by the addition of acid, which results in a color change to yellow. The optical absorbance of controls and specimens is determined with a spectrophotometer with wavelength set at 450 nm. Results of the test are interpreted by the absorbance value of the color change, based on a determined cutoff absorbance value. Specimens with absorbance values less than the cutoff are considered non-reactive or negative for the Human Immunodeficiency Virus. Those above the cutoff absorbance value are considered reactive or positive of Human Immunodeficiency Virus.

Cytomegalovirus

Cytomegalovirus (CMV) is a common human viral pathogen which belongs to the family of herpes viruses. The presence of CMV antibodies in an individual indicates prior infection by the virus. The test for CMV is a qualitative solid phase red cell adherence test system for the detection of antibodies (IgG plus IgM) to Cytomegalovirus (CMV) in human serum or plasma. A commercially-available test is the Capture CMV from Immuocor. Other suitable tests may be used to test for CMV.

The assay procedure is a two step solid phase red cell adherence test carried out in microtitration wells coated with inactivated CMV virus. Serum or plasma samples are added to the viral-coated wells. The specimen are incubated for five minutes; during which antibodies specific for CMV proteins bind to immobilized viral proteins. Unbound immunoglobulins are washed from the wells and replaced with a suspension of anti-IgG-plus anti-IgM-coated indicator red cells. Centrifugation brings the indicator red cells in contact with antibodies bound to the immobilized viral proteins. In the case of a positive test, the migration of the indicator red cells to the bottom of the wells is impeded as the anti-IgG and anti-IgM bridges are formed between the indicator red cells and the viral bound antibodies. As a consequence, the indicator red cells adhere over the surface of the microtitration well. In contrast, in the absence of viral antigen-antibody interactions (i.e., a negative test) the indicator red cells are not impeded during their migration, and pellet to the bottom of the well as a packed, well-defined cell button.

Human T-Cell Lymphotropic Virus (HTLV I/II)

Human T-cell Lymphotropic Virus (HTLV I/II) tests detects antibody to the HTLV-I and HTLV-II viruses. HTLV-I has been associated with adult T-cell leukemia/lymphoma (ATL) and HTLV-I is associated with myelopathy/tropical spastic paraparesis (HAM/TSP). The test for HTLV I/II is an in vitro enzyme immunoassay (EIA) for the qualitative detection of antibodies to Human T-Lymphotropic Virus Type I and Type II in human serum or plasma. A commercially-available test is the Inno-Lia HTLV I/II. Other suitable tests may be used to test for Human T-cell Lymphotropic Virus (HTLV I/II).

The assay utilizes a bead as a solid phase, coated with detergent-solubilized and sonicated HTLV proteins, to bind antibodies to the HTLV from human serum or plasma. Goat antibodies directed against human immunoglobulins, conjugated with horseradish peroxidase, are then incubated with the bead. Finally, the beads are incubated with o-phylenediamine (OPD) substrate solution containing hydrogen peroxide. A yellow-orange color develops if antibodies present in the sample bind to the bead.

The test specimen is diluted in specimen diluent and incubated with a polystyrene bead coasted with detergent solubilized HTLV-I and HTLV-II proteins (inactivated). Specific antibodies present in the sample bind to the HTLV-I and HTLV-II antigens on the bead. Unbound materials are removed by washing the beads. Goat antibody directed against human IgG that has been conjugated with horseradish peroxidase (anti-human IgG: HRPO) is then incubated with the beads and binds to the human IgG on the solid phase. Unbound conjugate is removed by washing the beads. The beads are then incubated with o-phenylenediamine (OPD) substrate solution containing hydrogen peroxide. The reaction of OPD substrate solution with HRPO yields a yellow-orange color. The intensity of the color formed is proportional to the amount of HTLV-I and/or HTLV-II antibody present in the sample. The enzyme reaction is stopped by the addition of sulfuric acid and the intensity of the color developed is read using a spectrophotometer. Results of the test are interpreted by the absorbance value of the color change, based on a determined cutoff absorbance value. Specimens with absorbance values less than the cutoff are considered non-reactive or negative for the Human T-Lymphotropic Virus. Those above the cutoff absorbance value are considered reactive or positive of Human T-Lymphotropic Virus.

West Nile Virus

West Nile Virus (WNV) is a mosquito-borne flavivirus that is associated with human disease ranging from mild flu-like symptoms to severe neurological degeneration. The test for WNV may be the commercially available Procleix WNV assay, which involves three main steps, occurring in a single tube: sample preparation; WNV RNA target amplification by transcription-mediated amplification (TMA); and detection of the amplification products by the hybridization protection assay. Other suitable tests may be used to test for West Nile Virus.

During sample preparation, RNA is isolated from specimens via the use of target capture. The specimen is treated with a detergent to solubilize the viral envelope, denature proteins and release viral genomic RNA. Oligonucleotides that are homologous to highly conserved regions of WNV are hybridized to RNA or DNA target, if present in the specimen, and captured onto magnetic microparticles that are separated from the specimen in a magnetic filed. Wash steps are utilized to remove extraneous components from the reaction tube. Magnetic separation and wash steps are performed with a target capture system.

Target amplification occurs via TMA, which is a transcription-based nucleic acid amplification method that utilizes two enzymes. Reverse transcriptase is used to generate a DNA copy. A polymerase then produces multiple copies of RNA from the DNA copy template. The Procleix WNV assay utilizes the TMA method to amplify regions of WNV RNA.

Detection is achieved by HPA, using single-stranded nucleic acid probes with chemiluminescent labels that are complementary to the amplicon. The labeled nucleic acid probes hybridize specifically to the amplicon. The selection reagent differentiates between hybridized and unhybridized probes by inactivating the label on unhybridized probes. During the detection step, the chemiluminescent signal produced by the hybridized probe is measured. Internal control is added to each test specimen, control, and assay calibrator via the working target capture reagent. A specimen is nonreactive, or negative, for the WNV if the signal is less than the cutoff. A specimen is reactive, or positive, for WNV if the signal is more than the cutoff.

Syphilis

The etiologic agent of Syphilis is the microorganism Treponema pallidum. Tests for syphilis detect antibody formed against Treponema pallidum by a human. The test reacts with non-treponemal lipid antigens. A commercially-available test is the fluorescent treponemal antibody absorption (FTA-ABS) test. Other suitable tests may be used to test for Syphilis.

The FTA-ABS test reaction detects circulating antibodies against the etiologic agent of syphilis. The primary reaction involves antibodies which attach to antigens along the surface and internal structure of the microorganism. This reaction occurs during the incubation step of the test while the serum or specimen is diluted 1:5 in sorbent and covers the smears of the microorganism. The sorbent is prepared from saprophytic Reiter treponeme culture which contains substances that remove non-specific antibodies to “group treponemal antigens” found in normal individuals, but does not significantly absorb the antibodies against the virulent treponema in the diseased population. A rinsing period follows the primary incubation, which removes all unbound serum antibody. A secondary reaction and incubation period then follows. The reagent used in the secondary reactions is a fluorescence labeled anti-human conjugate, which covers the smear. The antigen surface is then thoroughly rinsed free of unbound conjugate and viewed under an appropriate fluorescence microscope. Fluorescence intensity of patient serum is recorded relative to control standards which establish the specificity and sensitivity of the test procedure. The intensity of fluorescence is graded on a scale of 4+ to negative (no fluorescence).

Preliminary Study

A preliminary infectious disease study was performed on two menstrual flow specimens collected by a donor of menstrual fluid for menstrual stem cell collection and preservation. Established, licensed infectious disease testing methods were used for infectious diseases of interest and were employed by a third-party infectious disease testing facility. Menstrual flow comprising menstrual blood, fluid, cells and tissue was collected by the donor according to collection methods disclosed in U.S. patent application Ser. No. 12/074,423. The donor also provided a comparative, venous specimen of blood according to standard phlebotomy techniques for testing for infectious disease.

The menstrual flow samples were collected from the donor using individual procurement kits comprising media tubes in a rack covered in parafilm, at least one collection device and instructions for use (i.e., DivaCup), antiseptic cleaning pads, small plastic bags, small biohazard bag for shipment of specimens to laboratory, parafilm, nanocooler, and a collection form. The collection kit remained at a refrigerated temperature of about 1° C. to about 110° C. prior to use. The collection took place on one of the heaviest flowing days of the donor's menstrual cycle, which was usually the first and/or second day. The donor used a DIVA collection cup. The collection cup was sterilized prior to use, and the donor followed the manufacturer's instructions for use. Prior to collection, the donor washed her hands and used aseptic technique as much as possible during the collection of the menstrual flow.

The collection cup remained inside the vagina for less than about 3 hours to collect a menstrual flow sample for infectious disease testing. The donor removed the collection cup and poured the collected menstrual fluid specimen into about 10 milliliters of a isotonic solution (Hank's Balanced Salts Solution) with an anticoagulant (Heparin 10 units per milliliter) in a sterile 50 milliliter conical tube. The menstrual flow sample was poured into the conical tube while avoiding contact with the rim of the tube. The cap of the conical tube was screwed onto the conical tube and then was wrapped with a plastic parafilm to prevent leakage of the menstrual flow sample. The conical tube was inverted several times to mix the menstrual flow sample and the isotonic solution and anticoagulant.

The collection steps were repeated for the second sample. The first sample was refrigerated at a temperature of about 2° C. to about 8° C. until the second sample was collected. The conical tubes containing the menstrual flow samples and the venous sample were placed into a small plastic bag and in a thermal box with containing ice. The menstrual flow samples and venous sample were maintained at a temperature of about 1° C. to about 10° C. for the duration of shipment. The package containing the menstrual flow samples and venous sample were transported to the laboratory within at least about 48 hours of collection.

When the specimens arrived in the laboratory, each menstrual flow sample was separated into two EDTA-coated tubes. Each of the sets of two tubes were labeled and an order form was filled out to order infectious marker testing of the menstrual samples and a venous sample from the donor. The tests ordered included commercially-available infectious disease tests for Hepatitis B Surface Antigen, Hepatitis B Core Antibody, Hepatitis C Virus, Human Immunodeficiency Virus 1 & 2, Human T-Lymphotropic Virus I/II, Cytomegalovirus and Syphilis.

The results of the testing of the menstrual specimens showing comparative relationships for the preliminary test data is as follows.

TABLE 1
Infectious disease test results: Venous sample vs Menstrual
Sample - Qualitative and Quantitative Results
Specimen 1

| Test | Specimen1 - Venous | | Specimen1 - Menstrual | |
|---|---|---|---|---|
| HBsAg | Negative | Nonreactive | Negative | Nonreactive |
| HIV 1/2 | Negative | Nonreactive | Negative | Nonreactive |
| HBc | Negative | Nonreactive | Negative | Nonreactive |
| HTLV I/II | Negative | Nonreactive | Negative | Nonreactive |
| HCV | Negative | Nonreactive | Negative | Nonreactive |

Specimen 2

| | HBsAg | HBc | HCV | HIV I/2 | HTLV I/II |
|---|---|---|---|---|---|
| Venous | | | | | |
| Neg ctrl | 0.031 | 0.083 | 0.048 | 0.061 | 0.039 |
| Specimen ID74 | 0.018 | 0.047 | 0.009 | 0.056 | 0.061 |
| Pos ctrl | 1.378 | 1.365 | 1.696 | 1.575 | 0.693 |
| Cutoff value | 0.096 | 0.494 | 0.644 | 0.302 | 0.37 |
| Menstrual A | | | | | |
| Neg ctrl | 0.023 | 0.083 | 0.047 | 0.041 | 0.039 |
| Specimen ID75 | 0.029 | 0.052 | 0.014 | 0.028 | 0.042 |
| Pos ctrl | 1.378 | 1.365 | 1.71 | 1.265 | 0.727 |
| Cutoff value | 0.096 | 0.494 | 0.663 | 0.304 | 0.366 |
| Menstrual B | | | | | |
| Neg ctrl | 0.042 | 0.057 | 0.044 | 0.027 | 0.026 |
| Specimen ID618 | 0.055 | 0.076 | 0.006 | 0.042 | 0.034 |
| Pos ctrl | 1.38 | 1.09 | 1.749 | 1.494 | 0.856 |
| Cutoff value | 0.103 | 0.46 | 0.645 | 0.283 | 0.356 |

Qualitative Results, Specimen 2:

| Test | Results |
|---|---|
| HBsAg | Negative Nonreactive |
| HIV 1/2 | Negative Nonreactive |
| HBc | Negative Nonreactive |
| HTLV I/II | Negative Nonreactive |
| HCV | Negative Nonreactive |

Comparison Study for Peripheral Blood Samples and Menstrual Blood Samples

A study was performed using the methods of the present invention to analyze the infectious disease marker testing for Peripheral blood (PB) samples compared to menstrual blood (MB) samples which are also referred to as “M2” samples. 34 samples of paired PB and MB samples were tested. Menstrual flow specimens and corresponding peripheral blood samples were collected according to the present invention. Suitable volumes of menstrual flow specimens and peripheral blood specimens were tested or analyzed to determine the presence of infectious diseases using commercial infectious disease tests.

Infectious Disease Tests for the Comparison Study

The paired menstrual flow specimen and peripheral blood specimen were tested or analyzed for the presence of Hepatitis B Virus Core Antigen (anti-HBc) with the Ortho ELISA test system (Ortho (2006). Hepatitis B Virus Core Antigen (Recombinant). ELISA Test System. Raritan, N.J.) The testing comprised an Enzyme-linked immunosorbent assay (ELISA) in a three-stage test carried out in microwell coated with recombinant-derived hepatitis B core antigen (rHBcAg). In the first stage, each sufficient volume of test specimen of menstrual flow and peripheral blood was separately incubated in the test well with added diluent. During the second stage, an antibody conjugate specific for human IgG and IgM was added to the well. An enzyme detection system was added in the third stage. The detection system was comprised of hydrogen peroxide and o-phenylenediamine. Two paired peripheral blood and menstrual fluid samples tested positive to the HBc antigen, and the other paired peripheral blood and menstrual fluid samples tested negative for HBc antigen according to testing protocols. Exemplary data is shown in Table 2.

TABLE 2
Positive and Negative Results of Paired Testing
of PB/MB for Hepatitis B Virus Core Antigen

| Sample | HBc |
|---|---|
| M2-028IDT | 1.074 Reactive |
| PB-028IDT | 9.907 Reactive |
| M2-024IDT | 0.042 NR |
| M2-024IDT | 0.131 NR |

The paired menstrual flow specimen and peripheral blood specimen were tested or analyzed for the presence of Human T-Lymphotropic Virus Types I and II using the ABBOTT PRISM test (Abbott Laboratories (2007). Abbott Prism HTLV-I/HTLV-II. Abbott Park, Ill.). The ABBOTT PRISM System is an automated immunoassay analyzer for performing chemiluminescent immunoassays (ChLIA). Each paired menstrual flow and peripheral blood specimen were separately incubated with microparticles coated with sonicated and detergent-inactivated HTLV-I and HTLV-II antigens. The microparticles were then filtered out of the mixture and incubated with a probe consisting of biotinylated HTLV-I and HTLV-II proteins. Acridinium-labeled anti-biotin conjugate was added to the microparticles to bind any present probe. After incubation, the unbound conjugate was washed away. Alkaline hydrogen peroxide solution was added to generate any chemiluminescent signal. The resultant photons were counted. The amount of any light emitted is proportional to the amount of anti-HTLV-I and/or anti-HTLV-II in the sample. No paired peripheral blood and menstrual fluid samples tested positive for anti-HTLV-I and/or anti-HTLV-II. Exemplary data is shown in Table 3.

TABLE 3
Positive and Negative Result of Paired Testing of PB/MB
for Human T-Lymphotropic Virus Types I and II

| Sample | HTLV-1/II |
|---|---|
| M2-001IDT | 0.029 NR |
| PB-001IDT | 0.065 NR |

The paired menstrual flow specimen and peripheral blood specimen were tested or analyzed for the presence of Antibody to Hepatitis B surface antigen (mouse monoclonal IgM) using the ABBOTT PRISM system. Each paired menstrual flow and peripheral blood specimen were separately incubated with microparticles coated with mouse monoclonal anti-HBc. The microparticles were then filtered out of the mixture and incubated with acridinium-labeled goat polyclonal anti-HBc conjugate. After this incubation, the unbound conjugate was washed away. Alkaline hydrogen peroxide solution was added to generate a chemiluminescent signal. The resultant photons were counted. The amount of light emitted is proportional to the amount of HBsAg in the sample. One menstrual flow sample tested positive for HBsAg and its paired peripheral blood sample tested negative for HBsAg. All other paired samples tested negative for HBsAg. Exemplary data is shown in Table 4.

TABLE 4
Positive and Negative Result of Paired Testing
of PB/MB for Hepatitis B surface antigen

| Sample | HBsAg |
|---|---|
| M2-030IDT | Repeatedly Reactive |
| PB-030IDT | 0.069 NR |
| M2-039IDT | 0.057 NR |
| PB-039IDT | 0.030 NR |

The paired menstrual flow specimen and peripheral blood specimen were tested or analyzed for the presence of Hepatitis C Virus (Anti-HCV) using the ORTHO ELISA Test System (Ortho (2006). Hepatitis C Virus Encoded Antigen (Recombinant c22-2, c200, and NS5). ELISA Test System. Raritan, N.J.). The three-stage test was carried out in microwells coated with a combination of three recombinant hepatitis C virus (rHCV) antigens: c22-2, c200, and NS5. In the first stage, diluted specimens of each of the paired menstrual flow and peripheral blood specimens were incubated in the test well. During the second stage, murine monoclonal antibody conjugated to horseradish peroxidase were added to the wells. An enzyme detection system was added in the third stage and comprised hydrogen peroxide and o-phenylenediamine (OPD). The end-product was measured with a microwell reader. The amount of bound conjugate is proportional to the color intensity and is therefore a function of the concentration of anti-HCV present in the specimen. One paired menstrual flow specimen and peripheral blood specimen tested positive for HCV. All other paired samples tested negative. Exemplary data is shown in Table 5.

TABLE 5
Positive and Negative Result of Paired
Testing of PB/MB for Hepatitis C Virus

| Sample | HCV |
|---|---|
| M2-017IDT | 9.714 Reactive |
| PB-017IDT | 9.718 Reactive |
| M2-016IDT | 0.004 NR |
| PB-016IDT | 0.016 NR |

The paired menstrual flow specimen and peripheral blood specimen were tested or analyzed for the presence of Human Immunodeficiency Virus Types 1 and 2 using a BIO-RAD HIV-1/HIV-2 test (Bio-Rad (2007). Human Immunodeficiency Virus Types 1 and 2 (Recombinant Synthetic Peptides). Redmond, Wash.). The Bio-Rad test is an enzyme immunoassay based on the principle of the direct antibody sandwich technique. Solid phase microwell strip plates were coated with purified antigens: gp160 and p24 recombinant proteins derived from HIV-1; a peptide representing the immunodominant region of the HIV-2 transmembrane glycoprotein, gp36; and a synthetic polypeptide mimicking an artificial HIV-1 group O specific epitope. Paired menstrual flow and peripheral blood samples and controls were added to the microwells along with a specimen diluent containing a dye which changes color from purple to blue during this combination. The wells were incubated and washed. A green conjugate solution was added and the wells were incubated. The conjugate contains peroxidase-conjugated antigens (peptides mimicking various immunodominant epitopes of the HIV-1 and HIV-2 transmembrane glycoproteins, and a p24 recombinant protein). Working TMB solution was then added and allowed to incubate. A blue-green color develops in proportion to the amount of HIV antibody present in the sample. No paired samples tested positive for HIV-1 or HIV-2. Exemplary data is shown in Table 6.

TABLE 6
Positive and Negative Result of Paired Testing of PB/MB
for Human Immunodeficiency Virus Types 1 and 2

| Sample | HIV-1/II |
|---|---|
| M2-016IDT | 0.024 NR |
| PB-016IDT | 0.028 NR |

The paired menstrual flow specimen and peripheral blood specimen were tested or analyzed for the presence of Treponema pallidum using the OLYMPUS PK TP System (Olympus. Olympus PK TP System Microhemagglutination Test for Detection of Treponema pallidum Antibodies using the Olympus PK Instrument. Center Valley, Pa.). This system is based on the principle of agglutination and pattern recognition. Each of the paired menstrual flow and peripheral blood specimens were diluted in a diluent composed of phosphate-buffered saline containing normal rabbit testicular extract and cell components of sonicated Reiter T. phagedenis Sensitized cells were then added to a test mixture comprised of fixed chicken erythrocytes sensitized with components of the pathogenic T. pallidum. The reactants were allowed to settle in terraced microwells. Hemagglutination occurs in the presence of T. pallidum antibodies in the specimen. The OLYMPUS PK instrument was used to read the wells for agglutinated and unagglutinated patterns. A reactive test is a homogenous layer of cells; a nonreactive test would result in a compact dense button surrounded by a clear zone. No paired menstrual flow and peripheral blood specimens tested positive for Treponema pallidum. Exemplary data is shown in Table 7.

TABLE 7
Positive and Negative Result of Paired Testing
of PB/MB for Treponema pallidum (Syphilis)

| Sample | Syphilis |
|---|---|
| M2-001IDT | Neg/NR |
| PB-001IDT | Neg/NR |

The paired menstrual flow specimen and peripheral blood specimen were tested or analyzed for the presence of antibodies to Hepatitis B surface antigen (mouse monoclonal) using a BIO-RAD system (Bio-Rad (2007). Antibody to Hepatitis B Surface Antigen (Mouse Monoclonal). Redmond, Wash.). This system is a qualitative enzyme immunoassay for the detection of Hepatitis B surface antigen (HBsAG) in human serum or plasma. Wells of the microwell strip plates were coated with mouse monoclonal antibody to HBsAg (anti-HBs). Paired menstrual flow and peripheral blood specimens and appropriate controls were then added to the wells and incubated. Washing was followed by the addition of conjugate solution (peroxidase-conjugated mouse monoclonal antibodies directed against HBsAg). Working TMB Solution was added to the plate and incubated. A blue or blue-green color develops in proportion to the amount of HBsAG present in the sample.

The paired menstrual flow specimen and peripheral blood specimen were tested or analyzed using the HIV-1 Discriminatory Assay with the PROCLEIX ULTRIO system. The PROCLEIX ULTRIO Assay is a qualitative in vitro nucleic acid assay system for the detection of HIV-1 RNA, HCV RNA, and HBV DNA. The assay contains reagents which may be used for simultaneous detection of all three viruses or the individual viruses: HIV-1, HCV, and HBV. The first step of the assay was preparation of paired menstrual flow and peripheral blood specimens; in which viral RNA and DNA were isolated via the use of target capture. The specimens were treated with a detergent to solubilize the viral envelope, denature proteins and release viral genomic RNA and/or DNA. Oligonucleotides homologous to highly conserved regions of HIV-1, HCV, and HBV were hybridized to the HIV-1 RNA, HCV RNA, or HBV DNA target, if present, in the test specimens. The hybridized targets were then captured onto magnetic microparticles that were separated from the specimen in a magnetic field, and then washed. Target amplification occurred via TMA, a transcription-based nucleic acid amplification method that utilizes two enzymes, MMLV reverse transcriptase and T7 RNA polymerase. Reverse transcriptase was used to generate a DNA copy of the target sequence. The assay utilizes the TMA method to amplify regions of HIV-1 RNA, HCV RNA, and/or HBV DNA. Detection was achieved by HPA using single-stranded nucleic acid probes with chemiluminescent labels that are complementary to the amplicon. The labeled nucleic acid probes hybridized specifically to the amplicon. The selection reagent differentiates between hybridized and unhybridized probes by inactivating the label on unhybridized probes. During the detection step, the chemiluminescent signal produced by the hybridized probe was measured in a luminometer and was reported as Relative Light Units (RLU). Internal Control was added to each test specimen, control (if used), or assay calibrator tube via the working Target Capture Reagent that contains the Internal Control. The Internal Control in this reagent controls for specimen processing, amplification, and detection steps. Internal Control signal in each tube or assay reaction is discriminated from the HIV-1/HCV/HBV signal by the differential kinetics of light emission from probes with different labels. Internal Control-specific amplicon was detected using a probe with rapid emission of light (flasher signal). Amplicon specific to HIV-1/HCV/HBV is detected using probes with relatively slower kinetics of light emission (glower signal). The Dual Kinetic Assay (DKA) is a method used to differentiate between the signals from flasher and glower labels. When used for the simultaneous detection of HIV-1, HCV, and HBV, the assay differentiates between Internal Control and combined HIV-1/HCV/HBV signals but does not discriminate between individual HIV-1, HCV, and HBV signals. No paired menstrual flow or peripheral blood samples tested positive for any of the infectious diseases.

While preferred embodiments of the present invention have been shown and described, it will be apparent to those skilled in the art that many changes and modifications may be made without departing from the invention in its broader aspects. The appended claims are intended to cover, therefore, all such changes and modifications as fall within the true spirit and scope of the invention.

We claim:

1. A method for analyzing a non-venous and non-arterial puncture human fluid or cell sample to detect the presence of at least one infectious disease, the method comprising the steps of:
obtaining a sufficient volume of the non-venous and non-arterial puncture fluid or cell sample from a donor; and
testing the sufficient volume of the non-venous and non-arterial puncture human fluid or cell sample for an infectious disease.

2. The method of claim 1, wherein the method comprises the further steps of performing confirmatory testing of an arterial or venous blood sample from the donor and comparing confirmatory testing results to test results for the non-venous and non-arterial puncture human fluid or cell sample.

3. The method of claim 1, wherein the method comprises the further step of reporting test results.

4. The method of claim 1, wherein the non-venous and non-arterial puncture human fluid or cell sample is procured from any one of a specimen of menstrual fluid, endometrial menstrual cells, umbilical cord blood, or amniotic fluid.

5. The method of claim 1, wherein the infectious disease comprises any one of Hepatitis A, Hepatitis B, Hepatitis C, Cytomegalovirus, Human T-cell Lymphotropic Virus Type 1, Human T-cell Lymphotropic Virus Type II, Human Immunodeficiency Virus Type 1, Human Immunodeficiency Virus Type II, West Nile Virus, Trypansoma cruzi, Syphilis, and Treponema pallidum.

6. The method of claim 1, wherein the testing comprises a screening test or a confirmatory test.

7. The method of claim 1, wherein the testing comprises analyzing for an antigen or antibody associated with the presence of an infectious disease in a human.

8. A method for direct testing of a menstrual fluid sample from a human for the presence of at least one infectious disease, the method comprising the steps of:
isolating a sufficient volume of the menstrual fluid sample from a pre-collected specimen; and
analyzing the menstrual fluid sample for the presence of an infectious disease.

9. The method of claim 8, wherein the method comprises the further steps of performing confirmatory testing of an arterial or venous blood sample from the donor and comparing confirmatory testing results to test results for the menstrual fluid sample.

10. The method of claim 8, wherein the infectious disease comprises any one of Hepatitis A, Hepatitis B, Hepatitis C, Cytomegalovirus, Human T-cell Lymphotropic Virus Type 1, Human T-cell Lymphotropic Virus Type II, Human Immunodeficiency Virus Type 1, Human Immunodeficiency Virus Type II, West Nile Virus, Trypansoma cruzi, Syphilis, and Treponema pallidum.

11. The method of claim 8, wherein the testing comprises a screening test or a confirmatory test.

12. The method of claim 8, wherein the analyzing comprises determining the presence of an antigen or antibody associated with the presence of an infectious disease present in the menstrual fluid sample.

13. A method for direct testing of a human body fluid or cell sample for the presence of at least one infectious disease, the method comprising the step of;
preparing a sufficient volume of human body fluid or cell sample for analysis; and
analyzing human body fluid or cell sample for antigens or antibodies associated with an infectious disease.

14. The method of claim 13, wherein the method comprises the further steps of performing confirmatory testing of an arterial or venous blood sample from the donor and comparing confirmatory testing results to test results for the human body fluid or cell sample.
The method of claim 12, wherein the human body fluid or cell sample is procured from any one of a specimen of menstrual fluid, endometrial menstrual cells, umbilical cord blood, or amniotic fluid.

15. The method of claim 13, wherein the infectious disease comprises any one of Hepatitis A, Hepatitis B, Hepatitis C, Cytomegalovirus, Human T-cell Lymphotropic Virus Type 1, Human T-cell Lymphotropic Virus Type II, Human Immunodeficiency Virus Type 1, Human Immunodeficiency Virus Type II, West Nile Virus, Trypansoma cruzi, Syphilis, and Treponema pallidum.

16. The method of claim 13, wherein the testing comprises a screening test or a confirmatory test.

17. The method of claim 13, wherein the testing comprises analyzing for the presence and quantity of an antigen produced by an infectious disease or a human antibody associated with the presence of an infectious disease in a human.
